(19) United States
(12) Patent Application Publication
(10) Pub. No.: US 2026/0229317 A1
(43) Pub. Date: Aug. 6, 2026

(54) MACHINE LEARNING PLATFORM FOR ANTI-FLAMMABILITY MATERIALS

(71) Applicant: Polaron Analytics, Miamisburg, OH (US)

(72) Inventors: Duy Nhat Phan, Dayton, OH (US); Lokendra Poudel, Dayton, OH (US); Rahul Bhowmik, Centerville, OH (US)

(21) Appl. No.: 19/465,795

(22) Filed: Jan. 30, 2026

Related U.S. Application Data
(60) Provisional application No. 63/752,151, filed on Jan. 31, 2025.

(51) Int. Cl. G16C 20/30; G06N 20/20; G16C 20/70; G16C 20/90

(52) U.S. Cl. CPC G16C 20/30; G06N 20/20; G16C 20/70; G16C 20/90

(57) ABSTRACT

A process for creating a model for predicting flammability of materials for an application comprises creating or accessing a database of polymer descriptors and generating or accessing synthetic data corresponding to the polymer descriptors. Optimal descriptors for flammability parameters are determined. A model is then trained using synthetic data and then evaluated using data from the database of polymer descriptors and synthetic data.

### CROSS REFERENCE TO RELATED APPLICATIONS

This application claims the benefit of U.S. Provisional Patent Application Ser. No. 63/752,151, filed Jan. 31, 2025, having the title MACHINE LEARNING PLATFORM FOR ANTI-FLAMMABILITY MATERIALS, by Duy Nhat Phan et al., the entire disclosure of which is hereby incorporated herein by reference.

### STATEMENT REGARDING FEDERALLY SPONSORED DEVELOPMENT

This invention was made with government support under contract number N6833524C0124 awarded by U.S. Department of the Navy. The government has certain rights in the invention.

### BACKGROUND

Various aspects of the present disclosure relate generally to predictive models, and in particular to, predictive models for selection of anti-flammable materials.

A standard approach to measuring flammability involves destructive testing, such as cone calorimetry, which assesses properties like heat release rate, smoke production, and ignition time. These tests are highly reliable but have several drawbacks, including the high cost of equipment, the requirement for considerable sample quantities, and their time-consuming nature. They are also unsuitable for rapid or iterative testing, as each experiment consumes the material and requires preparation time.

### BRIEF SUMMARY

According to aspects of the present disclosure, a process for creating a model for predicting flammability of materials for an application comprises creating or accessing a database of polymer descriptors and generating or accessing synthetic data corresponding to the polymer descriptors. Optimal descriptors for flammability parameters are determined. A model is then trained using the synthetic data and then evaluated using data from the database of polymer descriptors and using synthetic data.

### BRIEF DESCRIPTION OF THE SEVERAL VIEWS OF THE DRAWINGS

FIG. 1 is a table showing ignition temperature, heat of combustion values, computed flammability index (FI) values, and FI labels by an expert and a machine learning model for various polymers of a polymer descriptor database, according to various aspects of the present disclosure;

FIG. 2 is a graph showing distribution of a flammability index for twenty-six polymers, according to various aspects of the present disclosure;

FIG. 3 is a graph illustrating distributions of synthetic data according to five flammability parameters, according to various aspects of the present disclosure;

FIG. 4 is a graph illustrating an optimal number of synthetic polymers using PDD descriptors required to provide an impact on the five flammability parameters, according to various aspects of the present disclosure;

FIG. 5 is a graph illustrating the top ten PDD (polymer descriptor database) descriptors in determining the five flammability parameters, according to various aspects of the present disclosure;

FIG. 6 is a graph illustrating an effect of the PDD polymer descriptors on the five flammability parameters, according to various aspects of the present disclosure;

FIG. 7 is a table illustrating results of random forest regression models using PDD descriptors to demonstrate strong predictive capabilities across the five flammability parameters, according to various aspects of the present disclosure;

FIG. 8 is a graph illustrating an optimal number of synthetic polymers using RDKit descriptors required to provide an impact on the five flammability parameters, according to various aspects of the present disclosure;

FIG. 9 is a graph illustrating the top ten RDKit descriptors in determining the five flammability parameters, according to various aspects of the present disclosure;

FIG. 10 is a graph illustrating an effect of the RDKit polymer descriptors on the five flammability parameters, according to various aspects of the present disclosure;

FIG. 11 is a table illustrating results of a random forest regression models using PDD and RDKit descriptors to demonstrate strong predictive capabilities across the five flammability parameters, according to various aspects of the present disclosure;

FIG. 12 is a table illustrating average results for both PDD and RDKit descriptors according to the five flammability parameters, according to various aspects of the present disclosure;

FIG. 13 is a flowchart illustrating a process for creating a machine-learning model for predicting capabilities of polymers across flammability metrics, according to various aspects of the present disclosure;

FIG. 14 is a screenshot of a module for use in an analytic tool accepting a .pdb file and a SMILES string, according to various aspects of the present disclosure;

FIG. 15 is a screenshot of the module displaying predicted flammability results after the submission of a .pdb file or SMILES string, according to various aspects of the present disclosure;

FIG. 16 is a screenshot of a visualization of the predicted flammability results, according to various aspects of the present disclosure; and

FIG. 17 is a block diagram of a computing system for operating the platform, the process, or both, according to various aspects of the present disclosure.

### DETAILED DESCRIPTION

According to aspects of the present disclosure, novel systems and processes for predicting polymer flammability properties through machine learning (ML), with the aim of providing accurate predictions of flammability index (FI), maximum heat release rate (pHRR), time to ignition (TIG), total smoke release, and fire growth rate is disclosed. The systems and processes combine a curated experimental dataset, synthetic data generation, and an accessible, cloud-based prediction tool, allowing researchers to input program database (pdb) files and simplified molecular input line entry system (SMILES) strings and receive flammability predictions through a user-friendly interface.

### Polymer Descriptor Database (PDD)

A custom PDD includes sixty-eight polymers with experimentally determined specific heat at constant pressure (C_p). For these polymers, ignition temperature (T_i) and heat of combustion (ΔH) values were extracted from existing literature. The following equation is used to calculate the flammability index (FI): FI=(C_p) (T_i)/ΔH.

For each polymer, one-hundred-eighty-eight descriptors (e.g., atom name, bonding, molecular weight, atoms, bonds, angles, dihedral, etc.) are extracted from the chemical structure (see Bhowmik, R.; Sihn, S.; Pachter, R.; Vernon, J. P. Prediction of the specific heat of polymers from experimental data and machine learning methods. Polymer 2021, 220, 123558, the entirety of which is hereby incorporated by reference). In addition to utilizing the polymer descriptor database (PDD), other descriptors may be used (e.g., descriptors generated by RDKit (https://www.rdkit.org), which is an open-source toolkit for cheminformatics).

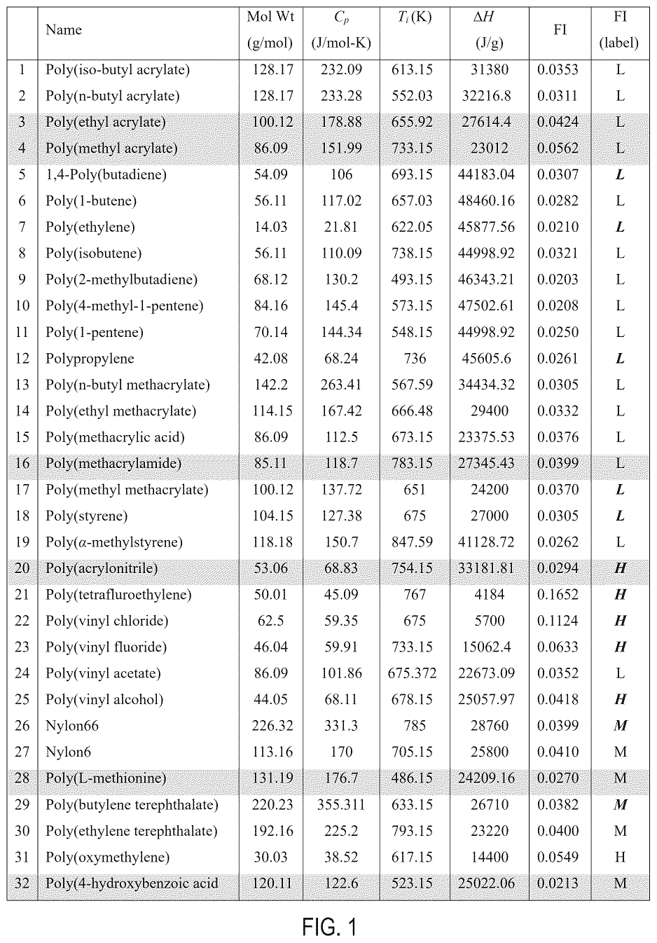

Ignition temperature values and heat of combustion values are extracted for thirty-two polymers out of the sixty-eight polymers in the PDD, as shown in FIG. 1. Those values are then used to determine a flammability index for each of the thirty-two polymers, as shown in the penultimate column of FIG. 1. Further, the polymer descriptor database categorized the polymers into three groups: low, medium, and high flammability index groups, as shown in the last column of FIG. 1. High (H) FI values are within the range of 0.0418 to 0.1652, the Medium (M) FI values are within the range of 0.0382 to 0.041, and the Low (L) FI values are within the range of 0.0203 to 0.0376.

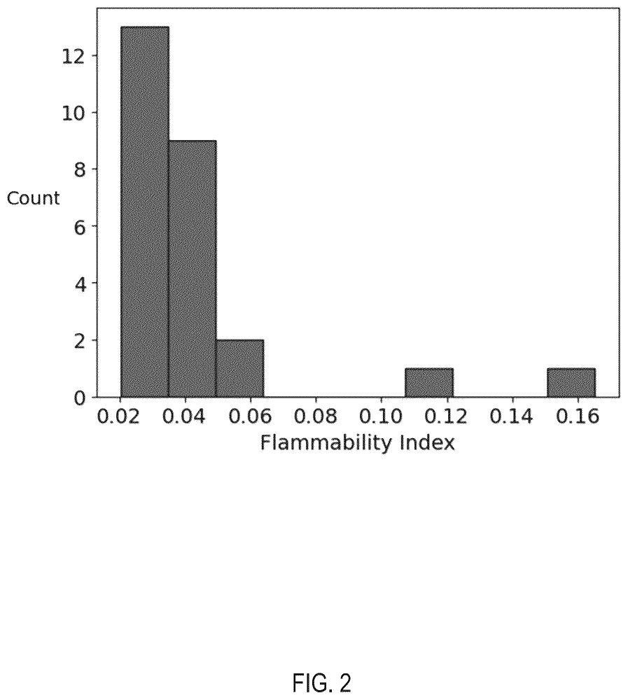

A Random Forest classification model was trained using the labeled polymers of FIG. 1 to predict flammability index categories for the remaining polymers. The FI Labels, indicated in a bolded and italicized font in the last column, are used to develop the Random Forest classification, while a non-bolded, non-italicized font represents the predicted labels. The predicted labels were subsequently used to identify and remove suspect flammability index values by comparing the FI values and labels to determine that the FI values correspond with their classified labels (i.e., FI group) in most cases. However, three polymers with high FI values have been classified as low, and three polymers with low FI values have been classified as medium or high. Specifically, polymers 3, 4, 16, 20, 28, and 32 (high-lighted gray in FIG. 1) were removed. The polymer rows highlighted in gray signify the incorrect FI values, as suggested by the trained ML model. Distribution of the flammability index of the remaining twenty-six polymers is presented in FIG. 2.

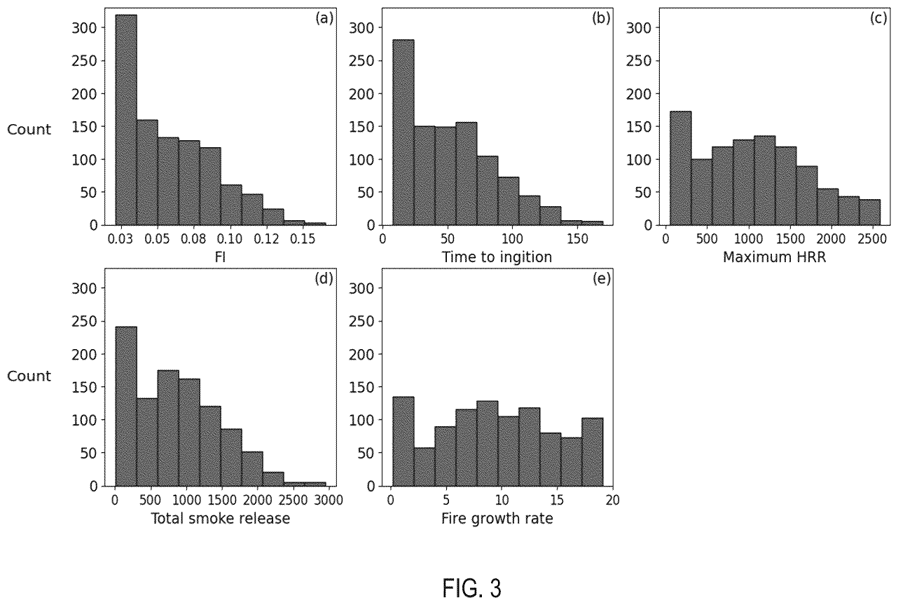

As applying ML approaches requires a substantial amount of training data to effectively recognize essential features for predictive tasks, and the results of the PDD data through the model are limited, the Synthetic Data Vault (SDV) was used to generate synthetic tabular data. SDV is an open-source Python library that utilizes a variety of machine learning algorithms, ranging from classical statistical methods to Generative AI, to learn patterns and relationships present in real data and emulate them in synthetic data. FIG. 3 illustrates the distributions of FI, TIG (ignition temperature), pHRR (peak heat release rate), total smoke release, and FIGRA (fire growth rate) for one-thousand synthetic polymers. Thus, synthetic data (see FI in FIG. 3) is used to flesh out the sparse real data (see FIG. 1) for use in training a model.

### Model Development

Random Forest regression models are used to predict the flammability index, ignition temperature, maximum heat release rate, total smoke release, and fire growth rate of polymers. Random forest regression, as a type of ensemble learning, combines the predictions of multiple decision trees to enhance predictive accuracy and mitigate overfitting, which is particularly effective in handling complex, non-linear relationships inherent in material properties. Hyperparameters such as the number of trees (estimators) and tree depth were optimized using cross-validation. The models' performance was evaluated using a R² score (defined below), with a robust assessment conducted through cross-validation. The R² score compares the predicted values generated by the models to the actual observed values, providing an indication of how well the models can generalize and predict the properties of testing polymers based on the knowledge acquired from synthetic polymers. An R² score higher than 0.7 (70%) is considered to indicate good predictive capabilities. The R² score is defined as follows

$${R}^{2} = 1 - \frac{{\sum }_{i = 1}^{n} {( {y}_{i} - {\hat{y}}_{i} )}^{2}}{{\sum }_{i = 1}^{n} {( {y}_{i} - \overline{y} )}^{2}}$$

where, y₁, y₂, . . . , yₙ are the actual (observed) values, ŷ₁, ŷ₂, . . . , ŷₙ are the predicted values, and y is the mean of the observed values.

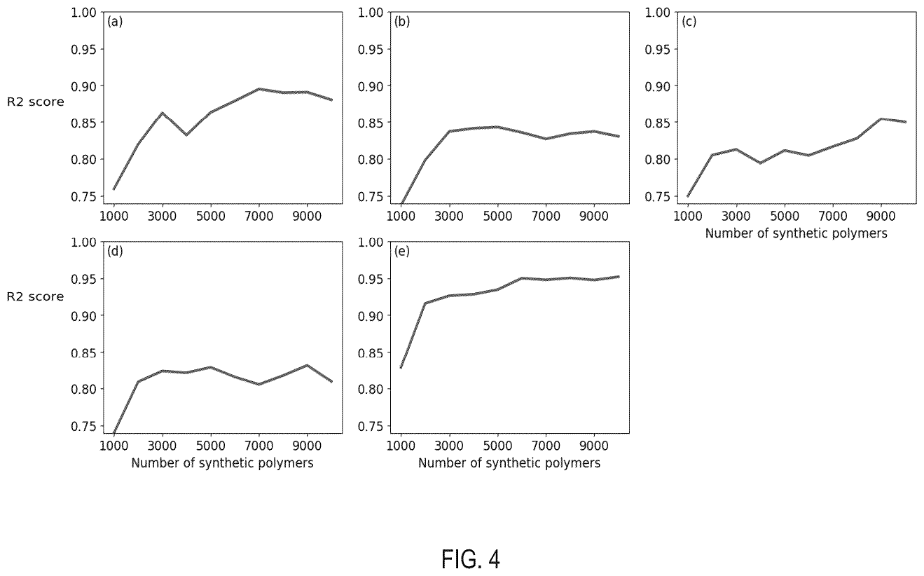

To assess an impact of the number of synthetic polymers (i.e., synthetic data) on model performance, models are trained using varying quantities of synthetic data, ranging from one-thousand to ten-thousand polymers. FIG. 4 displays the testing R² scores of the models, which indicate that models trained with data from seven-thousand, three-thousand, nine-thousand, five-thousand, and six-thousand synthetic polymers for predicting FI, TIG, pHRR, total smoke release, and FIGRA, respectively, achieved the highest testing R² scores of 0.89, 0.84, 0.85, 0.83, and 0.95 on the real polymers. In FIG. 4, the R² scores are shown for flammability parameters: FI (a), TIG (b), pHRR (c), total smoke release (d), and FIGRA (e) with respect to the number of synthetic polymers used for training. In FIG. 4, a point where the curve flattens is determined to be an optimal number of synthetic polymers used for each parameter: seven-thousand (a), three-thousand (b), nine-thousand (c), five-thousand (d), and six-thousand synthetic polymers (e).

### Descriptor Selection

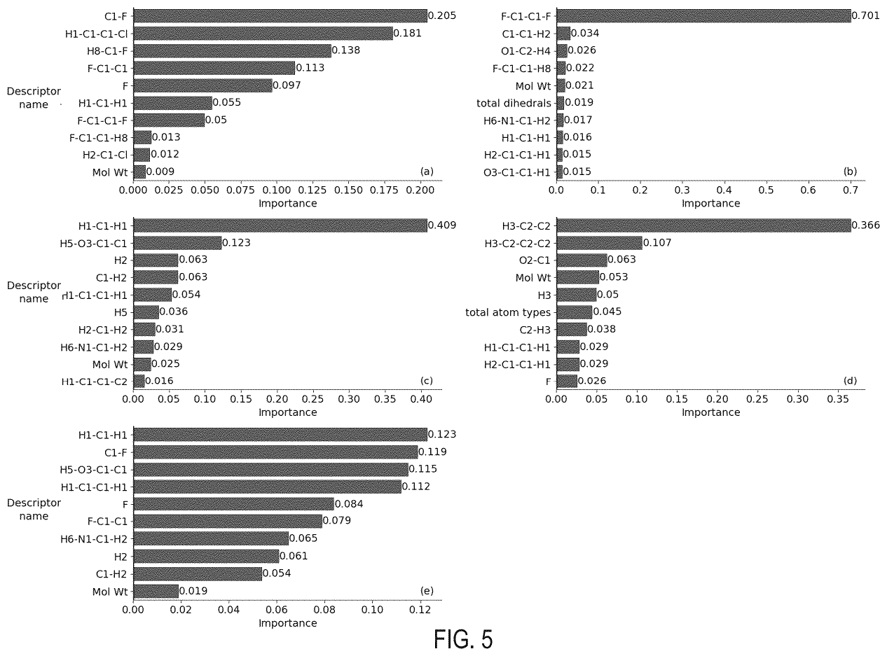

To identify which descriptors that significantly contribute to predicting flammability metrics, an importance of descriptors from models trained using the optimal number of synthetic polymers and 188 descriptors were evaluated, including assessing a contribution of each descriptor to reducing impurities (e.g., Gini impurity, entropy, etc.) during data splits. FIG. 5 illustrates the ten most important descriptors in FI (a), predicting TIG (b), predicting pHRR (c), predicting total smoke release (d), and predicting FIGRA (e).

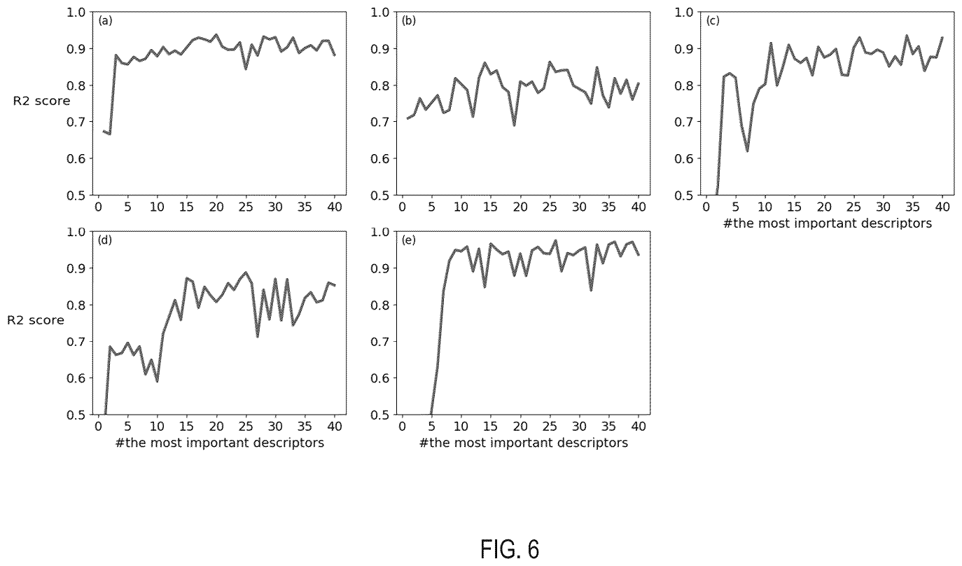

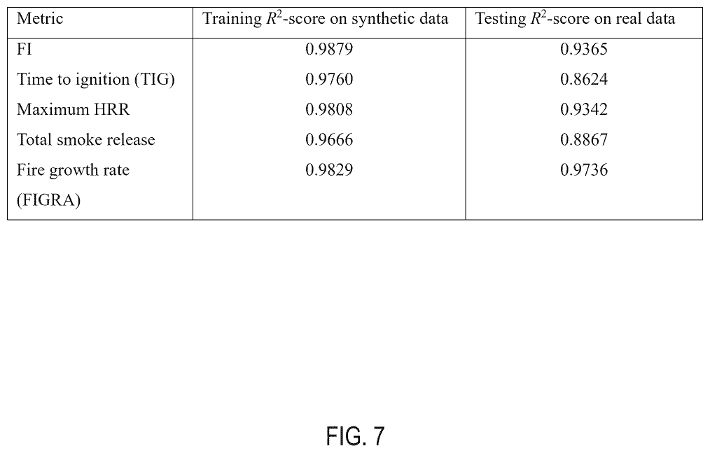

Further, FIG. 6 illustrates an effect of the polymer descriptors on flammability prediction (i.e., to predict flammability parameters (i.e., flammability metrics) of a polymer). Testing R² scores in predicting FI (a), predicting TIG (b), predicting pHRR (c), predicting total smoke release (d), and predicting FIGRA (e) with respect to the number of the most important descriptors. FIG. 7 is a table that illustrates training and testing of the models in predicting flammability metrics.

Based on the results in FIG. 7, the random forest regression models using PDD demonstrate strong predictive capabilities across flammability metrics. The training R² scores indicate an excellent model fit, with FI (0.98), time to ignition (0.97), maximum pHRR (0.98), total smoke release (0.96), and fire growth rate (0.98) all showing high accuracy. When evaluated on the testing set, the models maintained substantial predictive power, with FI and maximum pHRR both achieving an R² score of 0.93, total smoke release at 0.88, and fire growth rate at 0.97. Although the model's performance for time to ignition decreased to 0.86 in the testing phase, it still reflects a reliable level of accuracy. These results suggest that while the polymer descriptors provide robust model training, they also generalize well to new polymers, particularly for metrics like maximum pHRR and fire growth rate, making them effective for predicting flammability characteristics.

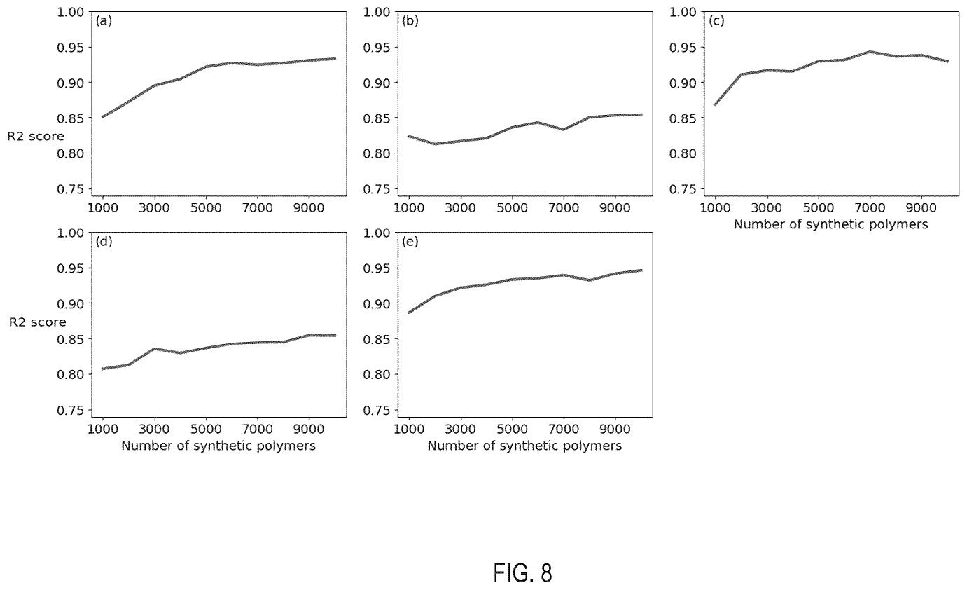

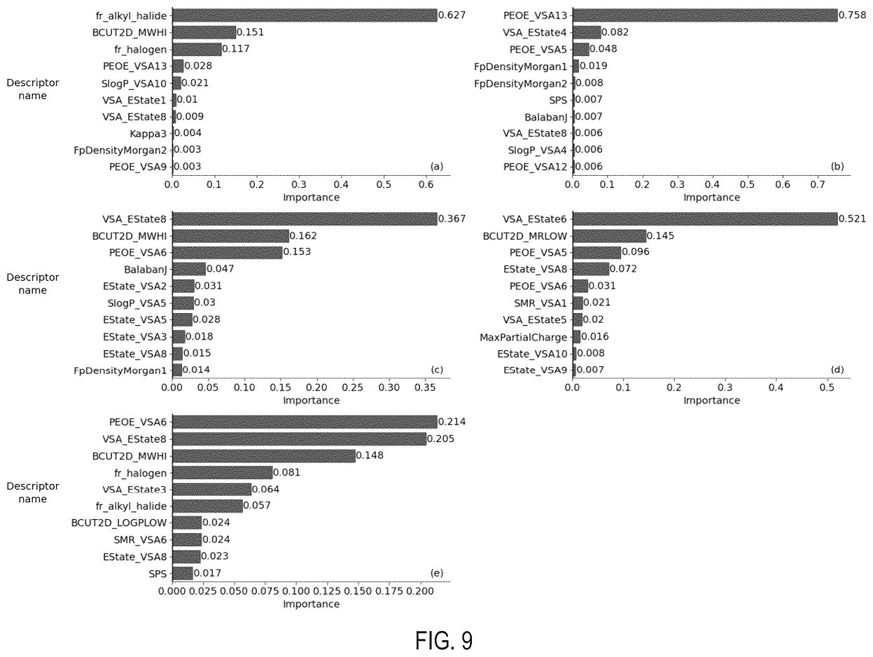

Exploring further descriptors, the RDKit (see above) libraries with two-hundred-and-ten descriptors were utilized in much the same manner as the descriptors from the PDD database. Similar to the PDD database, the RDKit includes descriptors such as a number of rotatable bonds, heavy atoms, hydrogen bond acceptors, hydrogen bond donors, molecular weight, etc. Applying the same procedure as above, an optimal number of synthetic polymers for training each model was determined as shown in FIG. 8. FIG. 8 illustrates R² scores in predicting FI (a), predicting TIG (b), predicting pHRR (c), predicting total smoke release (d), and predicting FIGRA (e) with respect to the number of synthetic polymers used for training. FIG. 9 illustrates the top ten important descriptors of the RDKit data for predicting flammability metrics: FI (a), predicting TIG (b), predicting pHRR (c), predicting total smoke release (d), and predicting FIGRA (e).

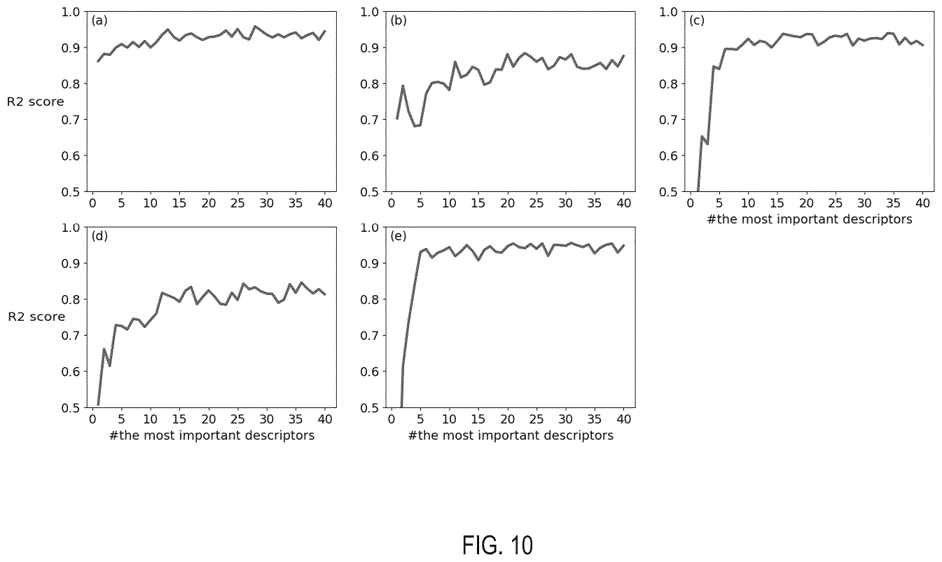

FIG. 10 illustrates an effect of RDKit descriptors on flammability prediction, where testing R² scores are shown in predicting FI (a), predicting TIG (b), predicting pHRR (c), predicting total smoke release (d), and predicting FIGRA (e) with respect to the number of the most important RDKit descriptors.

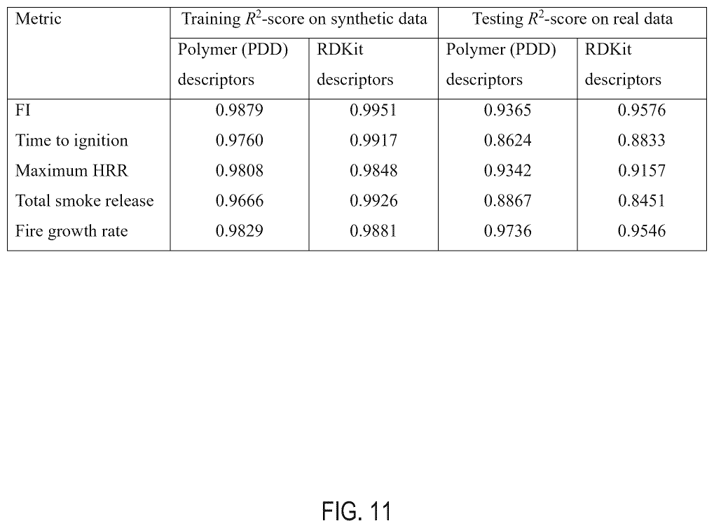

FIG. 11 summarizes performance of the random forest regression models using two different sets of descriptors (polymer (PDD) descriptors and RDKit descriptors) to predict flammability metrics, reported as R² scores for both training and testing datasets.

Evaluating a performance of random forest regression models using two sets of descriptors, RDKit and polymer (PDD), across various flammability metrics yielded results. During the training phase, models utilizing RDKit descriptors consistently outperformed those using polymer descriptors, as indicated by higher R² scores for Flammability Index, Time to Ignition, Maximum Heat Release Rate, Total Smoke Release, and Fire Growth Rate. However, in the testing phase, models with RDKit descriptors provided more accurate predictions for FI and Time to Ignition, while those with polymer descriptors excelled in predicting Maximum HRR, Total Smoke Release, and Fire Growth Rate. Importantly, the testing R² scores suggest that models using polymer descriptors may be more robust for certain metrics, potentially reducing the risk of overfitting on real polymers compared to those using RDKit descriptors.

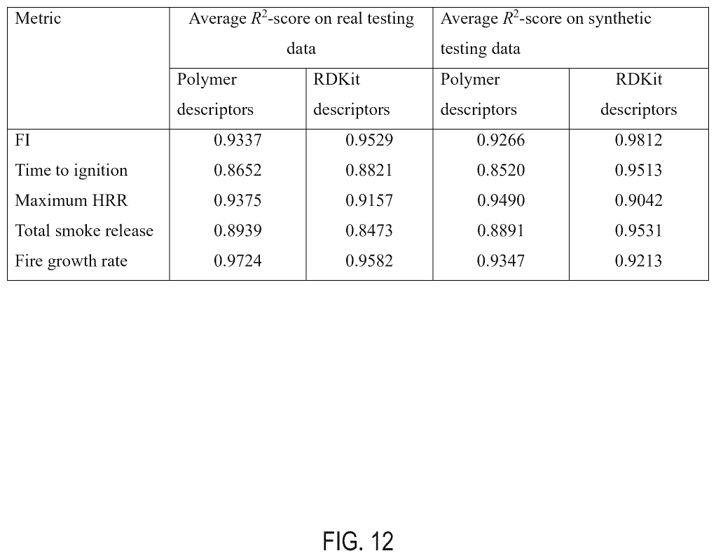

To avoid overfitting and provide a more realistic estimate of the model's generalization performance, a synthetic training set and a synthetic testing set were generated for each model. The models were on the training tests and evaluated on both synthetic testing sets and real testing sets. Average results for ten training and evaluations for each of the descriptor sets are shown in FIG. 12.

### Process

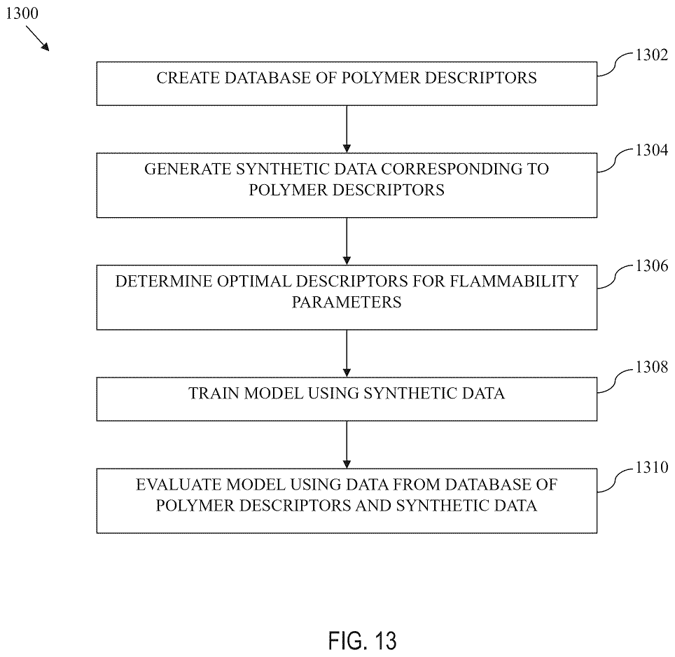

Turning to FIG. 13, a flow chart illustrating a process 1300 for creating a machine-learning model for predicting capabilities of polymers across flammability metrics. At 1302, a polymer descriptor database is created. For example, the PDD or the RDKit databases may be used as the database of descriptors. In some embodiments, polymers of the polymer descriptor database are grouped by a flammability parameter such as flammability index, as discussed above.

At 1304, synthetic data (as a number of synthetic polymers) is generated corresponding to the polymer descriptors. For example, if the database includes two-hundred descriptors, then the synthetic data should include two-hundred descriptors for each synthetic polymer of the synthetic data.

At 1306, optimal descriptors for flammability parameters are determined. For example, flammability parameters include FI, TIG, pHRR, total smoke release, and FIGRA, as discussed above. The optimal descriptors for each of the flammability parameters are determined by evaluating R² scores for each flammability parameter. For example, instead of using all two-hundred descriptors, only a small subset are optimal descriptors. The number of optimal descriptors may be a fixed number (e.g., 10), a number based on the total number of descriptors (e.g., 5% of the total number of descriptors), variable depending on a threshold (e.g., only when importance breaks an importance threshold for a given flammability parameter), other methods, or combinations thereof. In many embodiments, an optimal number of synthetic polymers (i.e., synthetic datasets) from the synthetic data is determined. The optimal number may be based on the flammability parameters, as discussed above.

Using the optimal descriptors, at 1308, a model is trained using the synthetic data, as discussed above. Then, at 1310, the model is evaluated using data from the database of polymer descriptors and synthetic data, as discussed, thus using a combined set of data. The resulting model may then be used to predict an optimal polymer for a given application based on the flammability parameters. The model enables rapid evaluation of multiple polymer formulations, allowing researchers to iterate on designs in a fraction of the time required for physical testing. By providing real-time predictions and insights, the model (and training and evaluation thereof) accelerates the material design process, supporting faster innovation cycles and helping industries bring safer, fire-resistant products to market more efficiently.

### User Interface and Interactive Development Tool

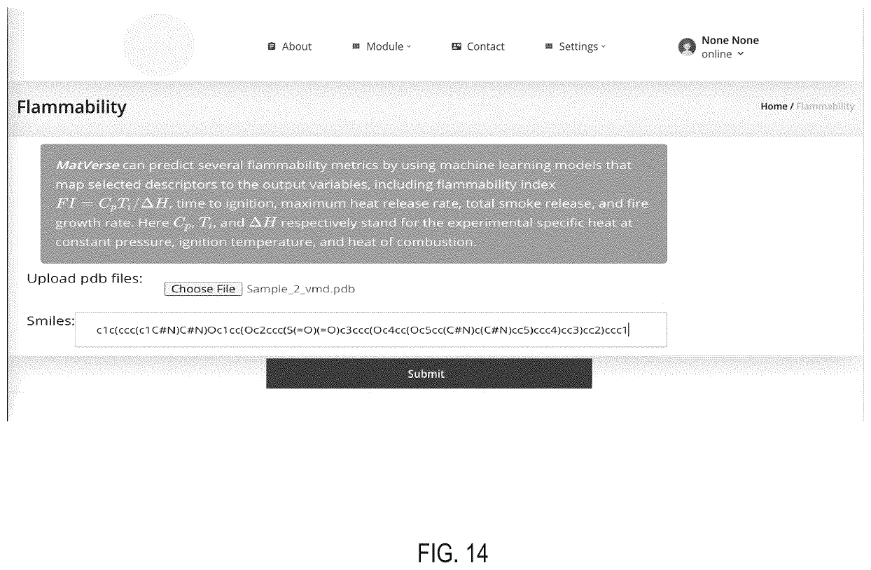

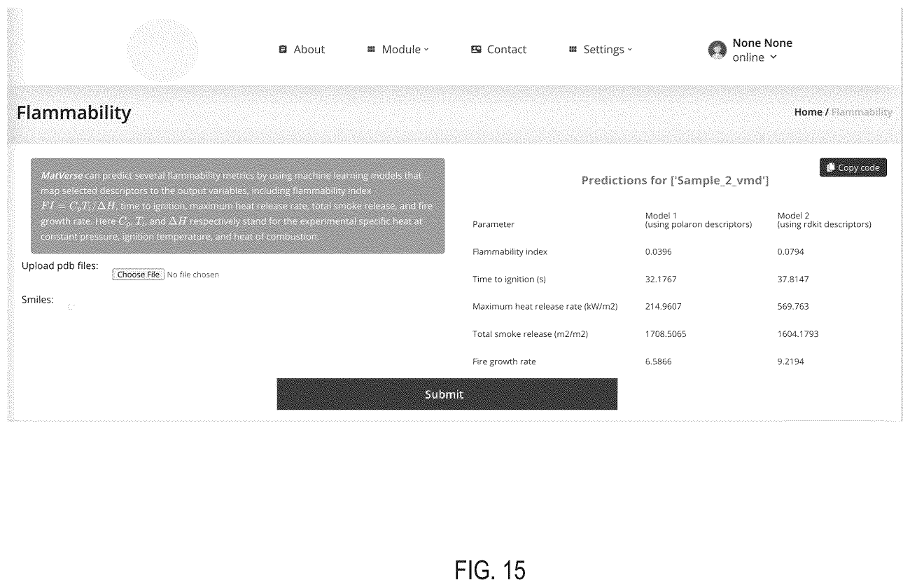

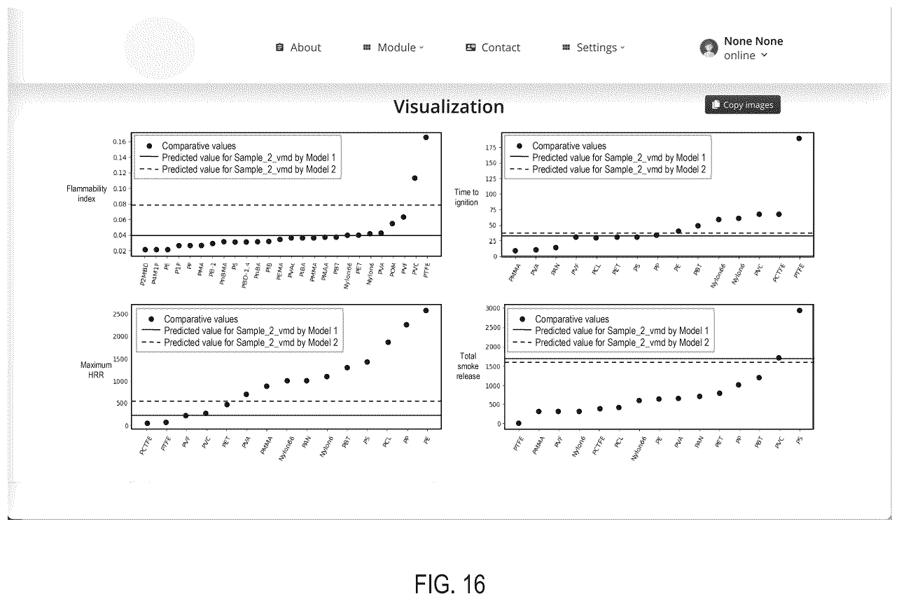

A module for use in an analytic tool includes descriptor databases and developed ML methods. Through a web-based interface, users can upload .pdb files and SMILES strings of polymers to predict flammability metrics of the polymers. Additionally, users can access visualizations comparing these predicted values with those in a standardized database. Screenshots of the module are illustrated in FIGS. 14-16. FIG. 14 illustrates the module accepting a .bpd file and a SMILES string. FIG. 15 illustrates the module displaying predicted flammability results after the submission of a .pdb file, SMILES string, or both. FIG. 16 shows a visualization of the predicted flammability results.

### Miscellaneous

An end-to-end framework for predicting polymer flammability using a unique combination of curated experimental data, synthetic data generation, and advanced machine learning models, all accessible through a user-friendly, cloud-based interface is disclosed. Traditional flammability testing methods are costly, destructive, and often inaccessible to many laboratories, creating a significant barrier to widespread fire-safe material innovation. The systems and processes described herein address these limitations by providing a robust, scalable alternative that enables accurate predictions of key flammability metrics, such as flammability index, maximum heat release rate, and time to ignition, without requiring extensive physical testing.

Further, the systems and processes herein use Synthetic Data Vault (SDV) to generate synthetic polymer data to overcome limitations posed by a scarcity of real-world experimental data, expanding the dataset and enhancing the machine learning model's robustness and generalizability. Thus, the systems and processes described herein ensure that the model can make reliable predictions for a diverse range of polymer compositions, even those not typically represented in experimental datasets. Thus, there is a practical solution to a longstanding challenge in polymer informatics: the lack of comprehensive, high-quality data for training predictive models.

Moreover, the systems and processes described herein include deep integration of polymer descriptors, drawn from an extensive Polymer Descriptor Database (PDD) and supplemented by additional descriptors generated with RDKit. This comprehensive selection captures nuanced chemical features that correlate closely with flammability behavior, enabling the model to make highly accurate predictions. Unlike many existing approaches, which rely on limited or generic descriptors, the systems and processes described herein have a depth in descriptor selection and categorization providing a highly detailed molecular profile, bringing greater accuracy and relevance to ML-driven polymer analysis.

Also, a module that embeds in an analysis program democratizes access to flammability predictions. By providing an intuitive, cloud-based interface, the module allows users, regardless of their technical expertise, to input polymer data, view predictions, and explore data visualizations that contextualize results alongside established materials in the database. This ease of use and accessibility are critical for expanding the practical utility of predictive modeling to a broader audience, including industry professionals, researchers, and product designers. The module's integration into a cloud environment removes technical barriers, making advanced predictive capabilities viable for users at all levels

Still further, the systems and processes described herein prioritize interpretability in predictive models. By providing insights into key polymer descriptors and enabling users to compare predicted and actual values, the systems and processes described herein enhance transparency in model predictions, an essential feature in safety-critical fields like fire safety engineering. This interpretability ensures that users can understand the basis of each prediction, adding a layer of confidence and reliability to the tool's outputs. Taken together, these features position the systems and processes described herein as pioneering solutions that bridge experimental polymer science with machine learning, facilitating faster, data-driven, and more accessible fire-safe material design

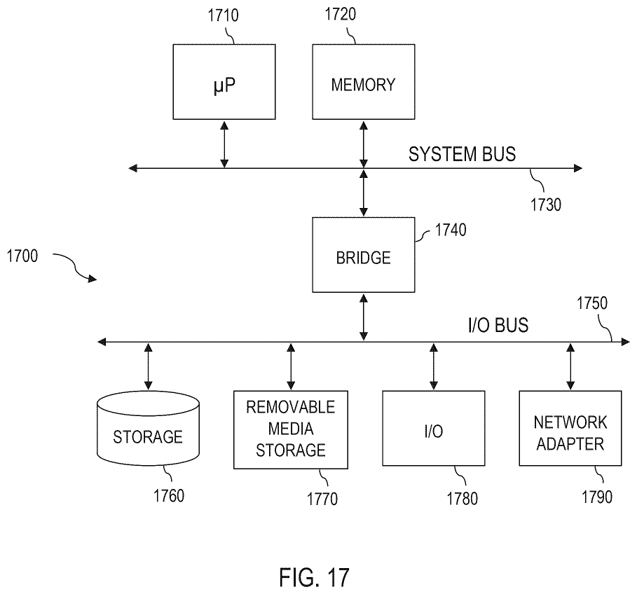

Referring to FIG. 17, a block diagram of a data processing system (i.e., computer system) is depicted in accordance with the present disclosure. Data processing system 1700 may comprise a symmetric multiprocessor (SMP) system or other configuration including a plurality of processors 1710 connected to system bus 1730. Alternatively, a single processor 1710 may be employed. Also connected to system bus 1730 is local memory 1720. An I/O bus bridge 1740 is connected to the system bus 1730 and provides an interface to an I/O bus 1750. The I/O bus may be utilized to support one or more buses and corresponding devices 1770, such as storage 1760, removable media storage 1770, input output devices (I/O devices) 1780, network adapters 1790, etc. Network adapters may also be coupled to the system to enable the data processing system to become coupled to other data processing systems or remote printers or storage devices through intervening private or public networks.

Also connected to the I/O bus may be devices such as a graphics adapter, storage and a computer usable storage medium having computer usable program code embodied thereon. The computer usable program code may be executed to implement any aspect of the present disclosure, for example, to implement any aspect of any of the methods and/or system components described herein.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present disclosure may take the form of a computer program product embodied in one or more computer readable storage medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM), Flash memory, an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device. A computer storage medium does not include propagating signals.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Network using an Network Service Provider).

Aspects of the present disclosure are described herein with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. Aspects of the disclosure were chosen and described in order to best explain the principles of the disclosure and the practical application, and to enable others of ordinary skill in the art to understand the disclosure for various embodiments with various modifications as are suited to the particular use contemplated.

What is claimed is:

1. A process for creating a model for predicting flammability of materials for an application, the process comprising:
creating a database of polymer descriptors;
generating synthetic data corresponding to the polymer descriptors;
determining optimal descriptors for flammability parameters;
training a model using the synthetic data; and
evaluating the model using a combined set of data from the database of polymer descriptors and synthetic data.

2. The process of claim 1, wherein generating synthetic data corresponding to the polymer descriptors further includes determining a number of synthetic datasets for each flammability parameter.

3. The process of claim 1, wherein creating a database of polymer descriptors includes grouping polymers according to a flammability parameter.

4. The process of claim 1, wherein determining optimal descriptors for flammability parameters includes evaluating R² scores for each flammability parameter.

5. The process of claim 1, wherein determining optimal descriptors for flammability parameters includes using a subset of all flammability parameters.

6. The process of claim 5, wherein determining optimal descriptors for flammability parameters includes using a subset of all flammability parameters, wherein a number of optimal descriptors for the flammability parameters is a fixed number.

7. The process of claim 5, wherein determining optimal descriptors for flammability parameters includes using a subset of all flammability parameters, wherein a number of optimal descriptors for the flammability parameters is a based on a total number of descriptors.

8. The process of claim 5, wherein determining optimal descriptors for flammability parameters includes using a subset of all flammability parameters, wherein a number of optimal descriptors for the flammability parameters is a variable based on a threshold.

9. The process of claim 1, wherein the flammability parameters include a flammability index (FI).

10. The process of claim 1, wherein the flammability parameters include a maximum heat release rate (pHRR).

11. The process of claim 1, wherein the flammability parameters include a time to ignition (TIG).

12. The process of claim 1, wherein the flammability parameters include total smoke release.

13. The process of claim 1, wherein the flammability parameters include a fire growth rate (FIGRA).

14. A process for creating a model for predicting flammability of materials for an application comprising:
accessing a database of polymer descriptors;
generating synthetic data corresponding to the polymer descriptors;
determining optimal descriptors for flammability parameters;
training a model using the synthetic data; and
evaluating the model using a combined set of data from the database of polymer descriptors and synthetic data.

15. The process of claim 14, wherein determining optimal descriptors for flammability parameters includes evaluating R² scores for each flammability parameter.

16. The process of claim 14, wherein determining optimal descriptors for flammability parameters includes using a subset of all flammability parameters.

17. The process of claim 16, wherein determining optimal descriptors for flammability parameters includes using a subset of all flammability parameters, wherein a number of optimal descriptors for the flammability parameters is a fixed number.

18. The process of claim 16, wherein determining optimal descriptors for flammability parameters includes using a subset of all flammability parameters, wherein a number of optimal descriptors for the flammability parameters is a based on a total number of descriptors.

19. The process of claim 16, wherein determining optimal descriptors for flammability parameters includes using a subset of all flammability parameters, wherein a number of optimal descriptors for the flammability parameters is a variable based on a threshold.

20. A process for creating a model for predicting flammability of materials for an application comprising:
accessing a database of polymer descriptors;
accessing synthetic data corresponding to the polymer descriptors;
determining optimal descriptors for flammability parameters;
training a model using the synthetic data; and
evaluating the model using a combined set of data from the database of polymer descriptors and synthetic data.
